(19) United States
(12) Patent Application Publication
(10) Pub. No.: US 2005/0100550 A1
(43) Pub. Date: May 12, 2005

(54) Anti-angiogenic uses of IL-6 antagonists

(76) Inventors: Mohit Trikha, Paoli, PA (US); Zhou Zhao, Collegeville, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/704,987

(22) Filed: Nov. 10, 2003

(51) Int. Cl. A61K 39/395

(52) U.S. Cl. 424/146.1; 424/155.1

(57) ABSTRACT

A method of using IL-6 antagonists to treat pathological processes associated with proliferative diseases, such as cancer, by specifically preventing or inhibiting the ability of new tissue to develop a blood supply. The invention more specifically relates to methods of treating such diseases by the use of IL-6 antagonists such as antibodies directed toward IL-6, including specified portions or variants, specific for at least one Interleukin-6 (IL-6 also known as interferon β2)) protein or fragment thereof, in an amount effective to inhibit angiogenesis.

### BACKGROUND OF THE INVENTION

Field of the Invention

The present invention relates to a method of using IL-6 antagonists to treat pathological processes associated with proliferative diseases, such as cancer, by specifically preventing or inhibiting the ability of new tissue to develop a blood supply. The invention more specifically relates to methods of treating such diseases by the use of IL-6 antagonists such as antibodies directed toward IL-6, including specified portions or variants, specific for at least one Interleukin-6 (IL-6 also known as interferon β2)) protein or fragment thereof, in an amount effective to inhibit angiogenesis.

Cytokine IL-6

IL-6 (interleukin 6) is a 22-27 kDa secreted glycoprotein formerly known as monocyte-derived human B-cell growth factor, B-cell stimulatory factor 2, BSF-2, interferon beta-2, and hybridoma growth factor, which has growth stimulatory and proinflammatory activities (Hirano et al. Nature 324: 73-76,1986).

IL-6 belongs to the granulocyte colony-stimulating factor (G-CSF) and myelomonocytic growth factor (MGF) family which includes leukemia inhibitory factor (LIF), oncostatin M (OSM), ciliary neurotropic factor (CNTF), cardiotropin-1 (CT-1), IL-1, and IL-11. IL-6 is produced by an array of cell types, most notably antigen presenting cells, T cells and B cells. IL-6-type cytokines all act via receptor complexes containing a common signal transducing protein, gp130 (formerly IL-6Rbeta). However, whereas IL-6, IL-11, CT-1, and CNTF bind first to specific receptor proteins which subsequently associate with gp130, LIF and OSMbind directly to a complex of LIF-R and gp130. The specific IL-6 receptor (IL-6R or IL-6alpha, gp80, or CD126) exists in either membrane bound or soluble forms (sIL-6R, a 55 kD form), which are both capable of activating gp130.

Several agents are known to induce the expression of IL-6 such as IL-1, IL-2, TNFα, IL-4, IFNα, oncostatin and LPS. IL-6 is involved in diverse activities such as B and T cell activation, hematopoiesis, osteoclast activity, keratinocyte growth, acute phase protein synthesis, neuronal growth and hepatocyte activation (Hirano et al. Int. Rev. Immunol;16(3-4):249-84,1998).

Although IL-6 is involved in many pathways, IL-6 knockout mice have a normal phenotype, they are viable and fertile, and show slightly decreased number of T cells and decreased acute phase protein response to tissue injury (Kopf M et al. (1994) Nature: 368(6469):339-42). In contrast, transgenic mice that over-express IL-6 develop neurologic disease such as neurodegeneration, astrocytosis, cerebral angiogenesis, and these mice do not develop a blood brain barrier (Campbell et al. PNAS 90: 10061-10065, 1993).

The Role of IL-6 in Cancer

IL-6 is implicated in the pathophysiology of several malignant diseases by a variety of mechanisms. IL-6 is hypothesized to be a causative factor in cancer-related morbidity such as asthenia/cachexia and bone resorption. Tumor-induced cachexia (Cahlin et al. (2000) Cancer Res; 60(19):5488-9), bone resorption and associated hypercalcemia were found to be diminished in IL-6 knockout mice (Sandhu et al. 1999). Cancer-associated depression, and cerebral edema secondary to brain tumors have also been associated with high levels of IL-6 (Musselman et al. Am J Psychiatry;158(8):1252-7, 2001). Multiple myeloma is malignancy involving plasma cells. IL-6 is known to enhance proliferation, differentiation and survival of malignant plasma cells in multiple myeloma (MM) through an autocrine or a paracrine mechanism that involves the inhibition of apoptosis of the malignant cells. Accordingly, blocking of IL-6 has been postulated to be an effective therapy (Anderson et al. Hematology:147-165, 2000) and clinical trials have been performed (Bataille et al. (1995) Blood; 86(2):685-91 and Van Zaanen, et al. (1996) J Clin Invest 98:1441-1448).

Experimental results from a number of in vitro and in vivo models of various human cancers have demonstrated that IL-6 is a therapeutic target for inhibition. IL-6 can induce proliferation, differentiation and survival of tumor cells, promote apoptosis (Jee et al. Oncogene 20: 198-208, 2001), and induce resistance to chemotherapy (Conze et al. Cancer Res 61: 8851-8858, 2001).

Squamous cell carcinoma is the most common malignancy of the larynx and of the head and neck. Often correlated to smoking the, common primary site is the vocal cords (particularly the anterior portion), epiglottis, pyriform sinus, and postcricoid area. Angiogenesis is correlated with regional recurrence (Ch. 88 The Merck Manual 17^{th} Ed. 1999). Kaposi's Sarcoma, common to HIV infected patients, is likely a vascular or dysplastic endothelial cell derived from a mesenchymal precursor cell, which may be transformed by exposure to an infectious agent. Activated KS cells produce IL-6, which functions as an autocrine factor to sustain the growth of the cells, and paracrine cytokines, which can stimulate proliferation of other mesenchymal cells and induce angiogenesis (Ch. 145 The Merck Manual 17^{th} Ed. 1999).

Monoclonal Antibodies to IL-6

Murine monocolonal antibodies to IL-6 are known as in, for example, U.S. Pat. No. 5,618,700. U.S. Pat. No. 5,856,135 discloses reshaped human antibodies to human IL-6 drived from a mouse monoclonal antibody SK2 in which the complementary determining regions (CDR's) from the variable region of the mouse antibody SK2 are transplanted into the variable region of a human antibody and joined to the constant region of a human antibody.

Another murine IL-6 monoclonal antibody referred to as CLB-6/8 capable of inhibiting receptor signaling was reported (Brakenhoff et al, J. Immunol. (1990) (145:561). A chimerized form of this antibody called cCLB8 was constructed (Centocor, Leiden, The Netherlands) and has been given to multiple myeloma patients (Van Zaanen, et al. 1996 supra). The method of making the resulting antibody from the murine antigen binding domains has been fully described in the applicants' copending application U.S. Ser. No. 60/332,743.

Analysis of patient serum samples prior to and after cCLB8 administration showed that circulating levels of both sIL6R and sgp130 were high in these patients and remained unchanged by the treatment despite total blockage of serum IL-6 activity (VanZaanen, et al. Leukemia Lymphoma 31(506): 551-558, 1998.)

B-E8 is a murine mAb to IL-6 manufactured by Diaclone, France which has also undergone clinical evaluation. B-E8 mAb demonstrated effectiveness in treating B-lymphoproliferative disorders (Haddad et al 2001). In AIDS associated lymphoma, this anti-IL-6 mAb had a clear effect on lowering lymphoma-associated fever and loss of weight due to cachexia, thereby improving indices of the quality of life for those patients (Emilie et al. (1994) Blood 84(8):2472-9). B-E8 has also been used in renal carcinoma patients. Metastatic renal cell carcinoma (RCC) is frequently associated with high levels of IL-6 and it is accompanied by paraneoplastic symptoms. B-E8 treatment had a significant reduction in the paraneoplastic syndrome in three RCC patients (Blay et al., Int J Cancer; 72(3): 424-30, 1997). In another published clinical trial, six patients with RCC were treated with B-E8 (Legouffe et al. (1994) Clin Exp Immunol. 98(2): 323-9). No obvious anti-tumor response was observed in these patients, however, all patients demonstrated a loss of symptoms generally attributable to IL-6 overproduction following B-E8 treatment.

The clinical experience with anti-IL6 Mabs has been limited to date. However, several in vitro and murine models of various human tumors have been used to demonstrate that anti-IL-6 Mabs have the potential to impact tumor cell survival and disease progression including: inhibiting growth of human brain tumor cells (Goswami et al. (1998) J Neurochem 71: 1837-1845) or tumors (Mauray et al. 2000), human renal carcinoma tumors and serum calcium concentrations (Weisglass et al. (1995) Endocrinology 138(5):1879-8), and human hormone refractory prostate tumor xenografts (Smith et al. (2001) Prostate; 48(1):47-53).

Disorders Associated with Inappropriate Angiogenesis

Angiogenesis is the process of generating new capillary blood vessels, and it results from activated proliferation of endothelial cells. Neovascularization is tightly regulated, and occurs only during embryonic development, tissue remodeling, wound healing and periodic cycle of corpus luteum development (Folkman and Cotran, Relation of vascular proliferation to tumor growth, Int. Rev. Exp. Pathol. '16, 207-248(1976)).

Endothelial cells normally proliferate much more slowly than other types of cells in the body. However, if the proliferation rate of these cells becomes unregulated, pathological angiogenesis can result. Pathological angiogenesis is involved in many diseases. For example, cardiovascular diseases such as angioma, angiofibroma, vascular deformity, atherosclerosis, synechia and edemic sclerosis; and opthalmological diseases such as neovascularization after cornea implantation, neovascular glaucoma, diabetic retinopathy, angiogenic corneal disease, macular degeneration, pterygium, retinal degeneration, retrolental fibroplasias, and granular conjunctivitis are related to angiogenesis. Chronic inflammatory diseases such as arthritis; dermatological diseases such as psoriasis, telangiectasis, pyogenic granuloma, seborrheic dermatitis, venous ulcers, acne, rosacea (acne rosacea or erythematosa), warts (verrucas), eczema, hemangiomas, lymphangiogenesis are also angiogenesis-dependent.

Vision can be impaired or lost because of various ocular diseases in which the vitreous humor is infiltrated by capillary blood. Diabetic retinopathy can take one of two forms, non-proliferative or proliferative. Proliferative retinopathy is characterized by abnormal new vessel formation (neovascularization), which grows on the vitreous surface or extends into the vitreous cavity. In advanced disease, neovascular membranes can occur, resulting in a traction retinal detachment. Vitreous hemorrhages may result from neovascularization. Visual symptoms vary. A sudden severe loss of vision can occur when there is intravitreal hemorrhage. Visual prognosis with proliferative retinopathy is more guarded if associated with severe retinal ischemia, extensive neovascularization, or extensive fibrous tissue formation. Macular degeneration, likewise takes two forms, dry and wet. In exudative macular degeneration (wet form), which is much less common, there is formation of a subretinal network of choroidal neovascularization often associated with intraretinal hemorrhage, subretinal fluid, pigment epithelial detachment, and hyperpigmentation. Eventually, this complex contracts and leaves a distinct elevated scar at the posterior pole. Both forms of age-related macular degeneration are often bilateral and are preceded by drusen in the macular region. Another cause of loss of vision related to angiogenic etiologies are damage to the iris. The two most common situations that result in the iris being pulled up into the angle are contraction of a membrane such as in neovascular glaucoma in patients with diabetes or central retinal vein occlusion or inflammatory precipitates associated with uveitis pulling the iris up into the angle (Ch. 99. The Merck Manual 17^{th} Ed. 1999).

Rheumatoid arthritis, an inflammatory disease, also results in inappropriate angiogenesis. The growth of vascular endothelial cells in the synovial cavity is activated by the inflammatory cytokines, and results in cartilage destruction and replacement with pannus in the articulation (Koch A K, Polverini P J and Leibovich S J, Arth; 15 Rhenium, 29, 471-479(1986); Stupack D G, Storgard C M and Cheresh D A, Braz. J. Med. Biol. Res., 32, 578-581(1999); Koch A K, Arthritis Rheum, 41, 951 962(1998)).

Psoriasis is caused by uncontrolled proliferation of skin cells. Fast growing cell requires sufficient blood supply, and abnormal angiogenesis is induced in psoriasis (Folkman J., J. Invest. Dermatol., 59, 40-48(1972)).

There is now considerable evidence that tumor growth and cancer progression requires angiogenesis, the formation of new blood vessels in order to provide tumor tissue with nutrients and oxygen, to carry away waste products and to act as conduits for the metastasis of tumor cells to distant sites (Folkman, et al. N Engl J Med 285: 1181-1186, 1971 and Folkman, et al. N Engl J Med 333: 1757-1763, 1995).

A number of factors are involved in processes and events leading to angiogenesis: cell adhesion molecules, integrins, vascular endothelial growth factor (VEGF), TNFalpha, bFGF, and cytokines including IL-6 and IL-12. For example, the closely related but distinct integrins aVb3 and aVb5 have been shown to mediate independent pathways in the angiogenic process. An antibody generated against aVb3 blocked basic fibroblast growth factor (bFGF) induced angiogenesis, whereas an antibody specific to aVb5 inhibited vascular endothelial growth factor (VEGF) induced angiogenesis (Eliceiri, et al., J. Clin. Invest.103: 1227-1230 (1999); Friedlander et al., Science 270: 1500-1502 (1995)). IL-6 is elevated in tissues undergoing angiogenesis and can induce VEGF in A431 cells, a human epidermoid carcinoma cell line (Cohen, et al. J. Biol. Chem. 271: 736-741, 1996).

IL-6 has been implicated in angiogenesis as a direct or indirect actor also because of its action on a number of cell types and observed expression in gonadotropin-primed hyperstimulated ovaries during a period of formation of a capillary network and vasculature extending fromm the ovarian medulla to growing follicles (Motro, B. et al. PNAS 87:3092-3096, 1990). IL-6 is a key factor in skin during injury and repair, and was shown to stimulate bovine brain endothelial cell migration which was inhibited by a neutralizing anti-IL-6 antibody (Rosen, et al. In: Cell Motility Factors. I.D. Goldberg, ed. Birkhaeuser Verlag, Basel. pp. 194-1205, 1991.)

Both the teratogenic and anti-tumor activity of thalidomide are believed linked to its anti-angiogenic activity. Thalidomide is reported to suppress levels of several cytokines including: TNFalpha, bFGF, VEGF, and IL-6. Another line of evidence for a role of IL-6 in tumor angiogenesis comes from data showing the stabilization of disease in renal cell carcinoma and some other types of cancer patients treated with thalidiomide. However, a correlation in TNFalpha, IL-6, bFGF, and VEGF levels and disease progression was not always significant (Eisen, et al. Br. J. Cancer 82:812-817, 2000 and Stebbing, et al. Br J Cancer 85: 953-958, 2001).

On the other hand, direct observation of vascular growth in an artificial tissue bed (MATRIGEL) inplanted in vivo showed that IL-6, IL-1 beta, PDGF were potent inhibitors of the neovascularization induced by fibroblast growth factors (Passanti, A. et al. Laboratory Invest. 67: 519-528, 1992).

In summary, IL-6 is a pleiotropic cytokine that can promote the pathogenesis of malignant diseases through several mechanisms. Preclinical data have shown that IL-6 is a survival, proliferation and differentiation factor in several types of tumors including renal cancer and prostate cancer. IL-6 also plays a major role in development of cancer related morbidity such as cachexia, bone resorption and depression and it can cause resistance to chemotherapy by inducing MDR1 gene expression. Clinical data have shown that elevated levels of IL-6 contribute to the malignant process in several diseases and preliminary clinical trials have shown some disease attenuating activity of anti-IL-6 Mabs, however, the association between IL-6 neutralization and a decrease in solid tumor growth or metastatic spread has not been made.

There is a long felt need for agents capable of limiting the growth and metastatic potential of a number of solid tumor types such as renal carcinoma and hormone refractory prostate carcinoma. Angiogenesis is know to be a contributing factor in number of pathological conditions including the ability of tumors to grow and metastasize, disorders of the eye including retinopathies, and disorders of the skin including Kaposi's Sarcoma. While numerous factors have been shown to be associated with these processes, including IL-6, it has not heretofore been demonstrated that an IL-6 antagonist with the ability to prevent IL-6 activation of receptor signaling has a direct effect on angiogenesis.

### SUMMARY OF THE INVENTION

The present invention relates to a method of using antagonists of IL-6, including antibodies directed toward IL-6, and specified portions or variants thereof specific for at least one Interleukin-6 (IL-6 also known as Interferon β2)) protein or fragment thereof, to inhibit angiogenesis in disease conditions associated with abnormal angiogenesis. Such anti-IL-6 antagonists such as antibodies can act through their ability to prevent the interaction of IL-6 with membrane bound receptor in a manner that prevents events associated with the initiation or progression of cancer tissue including events involved with angiogenesis, endothelial cell activation, and metastatic spread. Based on the aforementioned action of the IL-6 anatgonists of the invention, these antagonists can be best described as anti-angiogenic IL6 antagonists.

In a particular embodiment, the IL-6 antagonist is an antibody that specifically binds IL-6. A particular advantage of such antibodies is that they are capable of binding IL6 in a manner that prevents its action systemically. The antibodies may bind to IL6 creating a long-lived complex incapable of activating membrane bound receptor, such as gp130, in any tissue accessible by the complex through normal circulatory mechanisms. The method of the present invention thus employs antibodies having the desirable neutralizing property which makes them ideally suited for therapeutic and preventative treatment of metastatic disease states associated with various forms of cancer in human or nonhuman patients. Accordingly, the present invention is directed to a method of treating a disease or condition which is dependent on angiogenesis in a patient in need of such treatment which comprises administering to the patient an amount of a neutralizing IL-6 antibody to inhibit angiogenesis.

### BRIEF DESCRIPTION OF THE DRAWINGS

FIG. 1. A graph showing the data points representing the hemoglobin concentration measured in plugs injected into Nude mice with added human IL6. Each point represents one Matrigel plug (1 plug/animal), with the line representing the mean. A two-tailed unpaired t-test analysis calculated p<0.001 for all IL6 groups (compared to 0 ng/mL IL6).

FIG. 2: A graph showing the data points representing the hemoglobin concentration measured in plugs injected into nude mice with added murine IL6. Each point represents one Matrigel plug (1 plug/animal), with the line representing the mean. A two-tailed unpaired t-test analysis calculated p<0.001 for all IL6 groups (compared to 0 ng/mL IL6).

FIG. 3: A graph showing the data points representing the mean length of microvessels in plugs injected into nude mice with added human IL6. Each point represents average length/view from one Matrigel plug, with the line representing the mean of 10 plugs (2 plugs/animal). A two-tailed unpaired t-test analysis calculated p<0.001 for human IL6 groups compared to the control group (0 ng/mL IL6).

FIG. 4: A graph showing the data points representing the mean number of microvessels in plugs injected into nude mice with added human IL6. Each point represents average vessel number/view from one Matrigel plug, with the line representing the mean of 10 plugs (2 plugs/animal). A two-tailed unpaired t-test analysis calculated p<0.001 for human IL6 groups compared to the control group (0 ng/mL IL6).

FIG. 5. A graph showing the data points representing the mean length of microvessels in plugs injected into Nude mice with added murine IL6. Each point represents average length/view from one Matrigel plug, with the line representing the mean from 10 plugs (2 plugs/animal). A two-tailed unpaired t-test analysis calculated p<0.001 for murine IL6 groups compared to the control group (0 ng/mL IL6).

FIG. 6. A graph showing the data points representing the mean number of microvessels in plugs injected into nude mice with added murine IL6. Each point represents vessel number/view from one Matrigel plug, with the line representing the mean from 10 plugs (2 plugs/animal). A two-tailed unpaired t-test analysis calculated p<0.001 for murine IL6 groups compared to the control group (0 ng/mL IL6).

FIG. 7: A graph showing the data points representing the mean number of microvessels in plugs injected into nude mice with added human IL6 and with or without antibody. Each point represents microvessels per view from one Matrigel plug, with the line representing the mean of plugs (2 plugs/animal). A two-tailed unpaired t-test calculated p<0.001 for IL6-cCLB8 groups compared to IL6-C57 control group.

FIG. 8: A graph showing the data points representing the mean length of microvessels in plugs injected into nude mice with added human IL6 and with or without antibody. Each point represents average length per view from one Matrigel plug, with the line representing the mean of plugs (2 plugs/animal). A two-tailed unpaired t-test analysis calculated p<0.001 for IL6-cCLB8 groups compared to IL6-C57 control group.

FIG. 9: A graph showing the data points representing the hemoglobin concentration in Matrigel plugs. Each point represents one Matrigel plug, with the line representing the mean of plugs. Human IL6 was incorporated in the Matrigel at 200 ng/mL. A two-tailed unpaired t-test analysis calculated p<0.001 for all IL6-cCLB8 groups compared to IL6-C57 groups.

FIG. 10. A bar graph showing the effect of cCLB8 on IL6 induced apoptosis in human vascular endothelial cells. Data are mean±SD of triplicate determinations, expressed as percent of control (cells with no added hIL6).

FIG. 11. A graph showing the relationship between IL6 concentration and migration of HUVECs and U373 towards vitronectin in the presence of IL6. Each data point is the mean±SD of 3 measurements and all are relative to no added IL6.

FIG. 12. A bar graph showing that the migration of HUVECs towards vitronectin in the presence of IL6 can be attenuated by an anti-IL6 Mab, cCLB8. Each data point is the mean±SD of 3 migration transwell filters.

FIG. 13. A graph showing the ability of IL6 on HUVEC to enhance cell survival in serum free medium after 48 hrs. Data are mean±SD of triplicate determinations, expressed as percent increase in survival, with serum free medium without IL6 representing 0% increase in survival.

### DETAILED DESCRIPTION OF THE INVENTION

The anti-angiogenic IL-6 antagonists of the invention are useful in inhibiting and preventing angiogenesis in so far as they blocking the stimulatory effects of IL6 on endothelial cells, reduce endothelial cell division, decrease endothelial cell migration, and impair the activity of the proteolytic enzymes secreted by the endothelium. A number of pathologies including various forms of solid primary tumors and the metastases, lesions of the eye and disorders of the skin are improved by treatment with II-6 antagonists in the method of the present invention.

Cancer

Both benign and malignant tumors, including various cancers such as, cervical, anal and oral cancers, stomach, colon, bladder, rectal, liver, pancreatic, lung, breast, cervix uteri, corpus uteri, ovary, prostate, testis, renal, brain/cns (e.g., gliomas), head and neck, eye or ocular, throat, skin melanoma, acute lymphocytic leukemia, acute myelogenous leukemia, Ewing's Sarcoma, Kaposi's Sarcoma, basal cell carinoma and squamous cell carcinoma, small cell lung cancer, choriocarcinoma, rhabdomyosarcoma, angiosarcoma, hemangioendothelioma, Wilms Tumor, neuroblastoma, mouth/pharynx, esophageal, larynx, kidney and lymphoma, among others may be treated using anti-IL6 antibodies of the present invention. In addition, conditions such as neurofibromatosis, tuberous sclerosis (each of which conditions produces benign tumors of the skin), hemangiomas and lymphangiogenesis, among others, may be treated effectively with IL-6 antagonists according to the present invention.

A secondary tumor, a metastasis, is a tumor which originated in a primary site elsewhere in the body, but has now spread to a distant organ. The common routes for metastasis are direct growth into adjacent structures, spread through the vascular or lymphatic systems, and tracking along tissue planes and body cavaties with, for example, peritoneal fluid or cerebrospinal fluid. Secondary hepatic tumors are one of the most common causes of death in cancer patients and are by far and away the most common form of liver tumor. Although virtually any malignancy can metastasize to the liver, tumors which are most likely to spread to the liver include: cancer of the stomach, colon, and pancreas; melanoma; tumors of the lung, oropharynx, and bladder; Hodgkin's and non-Hodgkin's lymphoma; tumors of the breast, ovary, and prostate. Secondary lung, brain, and bone tumors are common to advanced stage breast, prostate and lung cancers. Any cancer may metastasize to bone, but metastases from carcinomas are the most common, particularly those arising in the breast, lung, prostate, kidney, and thyroid. Carcinoma of the lung is very commonly accompanied by hematogenous metastatic spread to the liver, brain, adrenals, and bone and may occur early, resulting in symptoms at those sites before obvious pulmonary symptom. Metastases to the lungs are common from primary cancers of the breast, colon, prostate, kidney, thyroid, stomach, cervix, rectum, testis, and bone and from melanoma. Each one of the above-named secondary tumors may be treated by the antibodies of the present invention.

In addition to tumors, numerous other non-tumorigenic angiogeneis-dependent diseases which are characterized by the abnormal growth of blood vessels may also be treated with the anti-angiogenic IL-6 antagonists of the present invention.

Representative examples of such non-tumorigenic angiogenesis-dependent diseases include corneal neovascularization, hypertrophic scars and keloids, proliferative diabetic retinopathy, rheumatoid arthritis, arteriovenous malformations (discussed above), atherosclerotic plaques, delayed wound healing, hemophilic joints, nonunion fractures, Osler-Weber syndrome, psoriasis, pyogenic granuloma, scleroderma, tracoma, menorrhagia (discussed above) and vascular adhesions.

Angiogenic Conditions of the Eyes

The cornea is a tissue which normally lacks blood vessels. In certain pathological conditions, however, capillaries may enter the cornea from the pericorneal vascular plexus of the limbus. When the cornea becomes vascularized, it also becomes clouded, resulting in a decline in the patient's visual acuity. Visual loss may become complete if the cornea completely opacitates.

Blood vessels can enter the cornea in a variety of patterns and depths, depending upon the process which incites the neovascularization. These patterns have been traditionally defined by ophthalmologists in the following types: pannus trachomatosus, pannus leprosus, pannus phylctenulosus, pannus degenerativus, and glucomatous pannus. The corneal stroma may also be invaded by branches of the anterior ciliary artery (called interstitial vascularization) which causes several distinct clinical lesions: terminal loops, a “brush-like” pattern, an umbel form, a lattice form, interstitial arcades (from episcleral vessels), and aberrant irregular vessels.

Corneal neovascularization can result from corneal ulcers. A wide variety of etiologies may produce corneal ulcers including for example corneal infections (trachoma, herpes simplex keratitis, leishmaniasis and onchocerciasis), immunological processes (graft rejection and Stevens-Johnson's syndrome), alkali burns, trauma, inflammation (of any cause), toxic and Vitamin A or protein deficiency states, and as a complication of wearing contact lenses.

While the cause of corneal neovascularization may vary, the response of the cornea to the insult and the subsequent vascular ingrowth is similar regardless of the cause. Several angiogenic factors are likely involved in this process, many of which are products of the inflammatory response. Indeed neovascularization of the cornea appears to only occur in association with an inflammatory cell infiltrate, and the degree of angiogenesis is proportional to the extent of the inflammatory reaction. Corneal edema further facilitates blood vessel ingrowth by loosening the corneal stromal framework through which the capillaries grow.

Topical therapy with IL-6 antibodies may also be useful prophylactically in corneal lesions which are known to have a high probability of inducing an angiogenic response (such as chemical burns). In these instances the treatment, likely in combination with steroids, may be instituted immediately to help prevent subsequent complications.

Such methods may also be utilized in a similar fashion to prevent capillary invasion of transplanted corneas. Use in combination with a steroid is also contemplated.

Neovascular glaucoma is a pathological condition wherein new capillaries develop in the iris of the eye. The angiogenesis usually originates from vessels located at the pupillary margin, and progresses across the root of the iris and into the trabecular meshwork. Fibroblasts and other connective tissue elements associate with the capillary growth and a fibrovascular membrane develops which spreads across the anterior surface of the iris eventually forming a scar. The scar formation prevents adequate drainage of aqueous humor resulting in an increase in intraocular pressure that may result in blindness.

Neovascular glaucoma generally occurs as a complication of diseases in which retinal ischemia is predominant. In particular, about one third of the patients with this disorder have diabetic retinopathy. Other causes include chronic retinal detachment, end-stage glaucoma, carotid artery obstructive disease, retrolental fibroplasia, sickle-cell anemia, intraocular tumors, and carotid cavernous fistulas.

Angiogenic Conditions of the Skin

Within another aspect of the present invention, methods are provided for treating hypertrophic scars and keloids, comprising the step of administering one of the above-described anti-angiogenic compositions to a hypertrophic scar or keloid.

Healing of wounds and scar formation occurs in three phases: inflammation, proliferation, and maturation. The first phase, inflammation, occurs in response to an injury which is severe enough to cause tissue damage and vascular leaking. During this phase, which lasts 3 to 4 days, blood and tissue fluid form an adhesive coagulum and fibrinous network which serves to bind the wound surfaces together. This is then followed by a proliferative phase in which there is ingrowth of capillaries and connective tissue from the wound edges, and closure of the skin defect. Finally, once capillary and fibroblastic proliferation has ceased, the maturation process begins wherein the scar contracts and becomes less cellular, less vascular, and appears flat and white. This final phase may take between 6 and 12 months.

Overproduction of connective tissue at the wound site causes a persistently cellular and possible red and raised scar to be formed. If the scar remains within the boundaries of the original wound it is referred to as a hypertrophic scar, but if it extends beyond the original scar and into the surrounding tissue, the lesion is referred to as a keloid. Hypertrophic scars and keloids are produced during the second and third phases of scar formation. Several wounds are particularly prone to excessive endlothelial and fibroblastic proliferation, including burns, open wounds, and infected wounds. With hypertrophic scars, some degree of maturation occurs and gradual improvement occurs. In the case of keloids however, an actual tumor is produced which can become quite large. Spontaneous improvement in such cases rarely occurs. Administration of an anti-IL-6 antibody in the method of the present invention to inhibit angiogenesis in such conditions can thus inhibit the formulation of such keloid scars.

Anti-Angiogenic Combinations with IL-6 Antagonists Such as Neutralizing Anti-IL6 Mabs

Angiogenesis is characterized by the invasion, migration and proliferation of smooth muscle and endothelial cells. The αᵥβ₃ integrin (also known as the vitronectin receptor) is known to play a role in various conditions or disease states including tumor metastasis, solid tumor growth (neoplasia), osteoporosis, Paget's disease, humoral hypercalcemia of malignancy, angiogenesis, including tumor angiogenesis, retinopathy, including macular degeneration, arthritis, including rheumatoid arthritis, periodontal disease, psoriasis and smooth muscle cell migration (e.g. restenosis).

The adhesion receptor integrin αᵥβ₃ binds vitronectin, fibrinogen, von Willebrand Factor, laminin, thrombospondin, and other like ligands. It was identified as a marker of angiogenic blood vessels in chick and man and plays a critical role in angiogenesis or neovascularization. Antagonists of αᵥβ₃ inhibit this process by selectively promoting apoptosis of cells in neovasculature. Therefore, αᵥβ₃ antagonists would be useful therapeutic targets for treating such conditions associated with neovascularization (Brooks et al., Science, Vol. 264, (1994), 569-571). Additionally, tumor cell invasion occurs by a three step process: 1) tumor cell attachment to extracellular matrix; 2) proteolytic dissolution of the matrix; and 3) movement of the cells through the dissolved barrier. This process can occur repeatedly and can result in metastases at sites distant from the original tumor. The αᵥβ₃ integrin has been shown to play a role in tumor cell invasion as well as angiogenesis.

As the antagonists of αᵥβ₃ and neutralizing anti-IL6 antibodies both target neovasculature but act through different mechanisms, the combination of anti-integrin antibodies with anti-IL6 antibodies should result in a particularly potent and effective combination therapy with little normal tissue toxicity. Thus, in one embodiment of the present invention, there is provided a method of treating a disease or condition associated with angiogenesis which comprises administering a combination of an integrin antagonist and an anti-IL-6 antibody to inhibit angiogenesis in a patient in need of such treatment. Other antibodies which selectively bind integrins or integrin subunits, especially those that bind the alphaV subunit, are disclosed in U.S. Pat. Nos. 5,985,278 and 6,160,099. Mabs that inhibit binding of alphaVbeta3 to its natural ligands containing the tripeptide argininyl-glycyl-aspartate (RGD) are disclosed in U.S. Pat. No. 5,766,591 and WO0078815.

A preferred combination of antibodies is the anti-alphaVbeta3 and anti-alphaVbeta5 Mab described in applicant's co-pending application U.S. Ser. No. 09/092,026 and an anti-IL-6 antibody referred to as cCLB8 disclosed in applicant's co-pending application Ser. No. 60/332,743. Both of the foregoing applications are incorporated by reference into the present application and form part of the disclosure hereof. In accordance with the invention, other known anti-angiogenesis agents such as thalidomide may also be employed in combination with an anti-IL-6 antibody.

Methods of Evaluating Anti-Angiogenic Activity

Widely accepted functional assays of angiogenesis and, hence, anti-angiogenic agents are the chick chorio-allantoic membrane assay (CAM) assay and the corneal micropocket assay of neovascularization.

For the CAM assay, fertilized chick embryos are removed from their shell on day 3 (or 4) and incubated in a Petri dish in high humidity and 5% CO2. On day 6, a methylcellulose disc (10 microL) containing the test substance is implanted on the chorioallantoic membrane. The embryos were examined 48 hours later, and if a clear avascular zone appears around the methylcellulose disc, the diameter of that zone is measured. The larger the zone, the more effective the antibody. India ink can be injected into the heart of some embryos just before formalin fixation so that vessels are visible near the edge of the avascular zone in histological sections. Histologic cross-sections of the chorioallantoic are examined to determine whether the test substance prevents normal development of the capillaries. This method, described in U.S. Pat. No. 5,001,116 which is also specifically incorporated herein by reference, showed the test useful in the selection of anti-angiogenic compounds or combinations of compounds.

The corneal micropocket assay of neovascularization may be practiced using rat or rabbit corneas. This in vivo model is widely accepted as being generally predictive of clinical effect, as described in many review articles and papers such as O'Reilly et. al. Cell 79: 315-328.

Briefly, a plug or pellet containing the recombinant bFGF (Takeda Pharmaceuticals-Japan) is implanted into corneal micropockets of each eye of an anesthetized female New Zealand white rabbit, 2 mm from the limbus followed by topical application of erythromycin ointment onto the surface of the cornea. The animals are dosed with the test compounds and examined with a slit lamp every other day by a corneal specialist. Various mathematical models are utilized to determine the amount of vascularized cornea and this formula was found to provide the most accurate approximation of the area of the band of neovascularization that grows towards the pellet.

The method may also be practiced using rats.

In the present invention, the corneal micropocket assay is used to demonstate the anti-angiogensis effect of anti-IL-6 antibodies. This is evidenced by a significant reduction in angiogenesis, as represented by a consistently observed and preferably marked reduction in the number of blood vessels within the cornea. Such responses are preferably defined as those corneas showing only an occasional sprout and/or hairpin loop that displayed no evidence of sustained growth when contacted with the test substance.

Endothelial and Non-Endothelial Cell Proliferation

It is important to establish which cell types are involved in the angiogenic processes specific for tumor vascularization. Tumor vessels as generally primitive, that is, contain only endothelial cells. Other cell types include: endothelial cells, smooth muscle cells, retinal pigment epithelial cells, fibroblasts, and epithelial cells, as well as tumor cells such as hemangioendothelioma cells or carcinoma cells. One example of an angiogenesis inhibitor that specifically inhibits endothelial cell proliferation is ANGIOSTATIN® protein. (O'Reilly et al., 1994 supra).

Various representative cell lines are available for testing. Bovine aortic smooth muscle (SMC), bovine retinal pigment epithelial (RPE), mink lung epithelial (MLE), Lewis lung carcinoma (LLC), and EOMA hemangioendothelioma cells and 3T3 fibroblasts. For the proliferation assays, cells are washed with PBS and dispersed in a 0.05% solution of trypsin. Optimal conditions for the cell proliferation assays are established for each different cell type. Generally, cells are trypsinized and re-seeded in growth medium in the presence and absence of IL6 and anti-IL6 neutralizing Mab. After approximately 72 hours, the change in cell number is assessed as by using a vital stain such as a tetrazolium dye base assay or by LDH release (Promega, Madison Wis.) or can are dispersed in trypsin, resuspended and counted by hand or using an automated device such as a Coulter counter.

IL-6 Antagonists

As used herein, the term “IL-6 antagonists” refers to a substance which inhibits or neutralizes the angiogenic activity of IL-6. Such antagonists accomplish this effect in a variety of ways. One class of IL-6 antagonists will bind to IL-6 protein with sufficient affinity and specificity to neutralize the angiogenic effect of IL-6. Included in this class of molecules are antibodies and antibody fragments (such as for example, F(ab) or F(ab′)₂ molecules). Another class of IL-6 antagonists are fragments of IL-6 protein, muteins or small organic molecules i.e. peptidomimetics, that will bind to IL-6, thereby inhibiting the angiogenic activity of IL-6. The IL-6 antagonist may be of any of these classes as long as it is a substance that inhibits IL-6 angiogenic activity. IL-6 antagonists include IL-6 antibody, IL-6R antibody, an anti-gp130 antibody or antagonist, modified IL-6 such as those disclosed in U.S. Pat. No. 5,723,120, antisense IL-6R and partial peptides of IL-6 or IL-6R.

Anti-IL-6 Antibodies

Any of the anti-IL-6 antibodies known it the art may be employed in the method of the present invention. Murine monocolonal antibodies to IL-6 are known as in, for example, U.S. Pat. No. 5,618,700. U.S. Pat. No. 5,856,135 discloses reshaped human antibodies to human IL-6 derived from a mouse monoclonal antibody SK2 in which the complementary determining regions (CDR's) from the variable region of the mouse antibody SK2 are transplanted into the variable region of a human antibody and joined to the constant region of a human antibody.

Another murine IL-6 monoclonal antibody referred to as CLB-6/8 capable of inhibiting receptor signaling was reported (Brakenhoff et al, J. Immunol. (1990) (145:561). A chimerized form of this antibody called cCLB8 was constructed (Centocor, Leiden, The Netherlands) and has been given to multiple myeloma patients (Van Zaanen, et al. 1996 supra). The method of making the resulting antibody from the murine antigen binding domains has been fully described in the applicants' copending application U.S. Ser. No. 60/332,743, hereby incorporated by reference into the present application.

Compositions and Their Uses

The neutralizing anti-IL6 monoclonal antibody described herein can be used to inhibit angiogenesis and thus prevent or impair tumor growth and prevent or inhibit metastases. Additionally, said monoclonal antibody can be used to inhibit angiogenic inflammatory diseases amenable to such treatment, which may include but are not limited to rheumatoid arthritis, diabetic retinopathy, psoriasis, and macular degeneration. The individual to be treated may be any mammal and is preferably a primate, a companion animal which is a mammal and most preferably a human patient. The amount of monoclonal antibody administered will vary according to the purpose it is being used for and the method of administration.

The anti-angiogenic anti-IL6 antibodies of the invention of the present invention may be administered by any number of methods that result in an effect in tissue in which angiogenesis is desired to be prevented or halted. Further, the anti-antiangiongenic anti-IL6 antibodies of the invention need not be present locally to impart and anti-angiogenic effect, therefore, they may be administered wherever access to body compartments or fluids containing IL6 is achieved. In the case of inflamed, malignant, or otherwise compromised tissues, these methods may include direct application of a formulation containing the antibodies. Such methods include intravenous administration of a liquid composition, transdermal administration of a liquid or solid formulation, oral, topical administration, or interstitial or inter-operative administration. Adminstration may be affect by the implantation of a device whose primary function may not be as a drug delivery vehicle as, for example, a vascular stent.

In particular, within one aspect of the present invention methods are provided for treating corneal neovascularization (including corneal graft neovascularization), comprising the step of administering a therapeutically effective amount of an anti-angiogenic anti-IL6 antibodies of the invention directly to the cornea or systemically to the patient, such that the formation of blood vessels is inhibited.

Within another aspect of the present invention methods are provided for treating neovascular glaucoma, comprising the step of administering a therapeutically effective amount of an anti-angiogenic neutralizing anti-IL6 antibodies directly to the eye or systemically to the patient, such that the formation of blood vessels is inhibited.

In another embodiment of the present invention either an anti-angiogenic anti-IL6 antibody of the invention alone, or in combination with another anti-angiogenic agent are directly injected into a hypertrophic scar or keloid in order to prevent the progression of these lesions. This therapy is of particular value in the prophylactic treatment of conditions which are known to result in the development of hypertrophic scars and keloids such as burns. Therapy may be effective when begun after the proliferative phase has had time to progress (approximately 14 days after the initial injury), but before hypertrophic scar or keloid development.

Administration may also be oral or by local injection into a tumor or tissue but generally, the monoclonal antibody is administered intravenously. Generally, the dosage range is from about 0.05 mg/kg to about 12.0 mg/kg. This may be as a bolus or as a slow or continuous infusion which may be controlled by a microprocessor controlled and programmable pump device.

Alternatively, DNA encoding preferably a fragment of said monoclonal antibody may be isolated from hybridoma cells and administered to a mammal. The DNA may be administered in naked form or inserted into a recombinant vector, e.g., vaccinia virus in a manner which results in expression of the DNA in the cells of the patient and delivery of the antibody.

The monoclonal antibody used in the method of the present invention may be formulated by any of the established methods of formulating pharmaceutical compositions, e.g. as described in Remington's Pharmaceutical Sciences, 1985. For ease of administration, the monoclonal antibody will typically be combined with a pharmaceutically acceptable carrier. Such carriers include water, physiological saline, or oils.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. Except insofar as any conventional medium is incompatible with the active ingredient and its intended use, its use in any compositions is contemplated.

The formulations may be presented in unit-dose or multi-dose containers, for example, sealed ampules and vials, and may be stored in a freeze-dried (lyophilized) condition requiring only the addition of the sterile liquid carrier, for example, water for injections, immediately prior to use.

Abbreviations

Abs antibodies, polyclonal or monoclonal
aV integrin subunit alpha V
b3 integrin subunit beta 3
bFGF basic fibroblast growth factor
IFN interferon
Ig immunoglobulin
IgG immunoglobulin G
IL interleukin
IL6 interleukin 6
IL-6R interleukin-6 receptor
sIL-6R soluble interleukin-6 receptor
Mab monoclonal antibody
VEGF vascular endothelial growth factor

While having described the invention in general terms, the embodiments of the invention will be further disclosed in the following examples.

### EXAMPLE 1

### Demostration of IL6 Induced Angiogenesis In Vivo

In Study 1, the primary angiogenic stimulus was IL6 added to the Matrigel plug. The mice received a subcutaneous injection of Matrigel with and without human or murine IL6 (Table 1). Matrigel forms a gelatinous plug once it reaches body temperature and this plug is stable within the body of the animal.

TABLE 1
The design for Study 1 in which 55 mice were
randomized into 11 groups (n = 5/group).

| | | |
|---|---|---|
| | | IL6 in Matrigel |
| Group # | n | (ng/mL) |

| | | | |
|---|---|---|---|
| 1 | 5 | — | 0 |
| 2 | 5 | Human | 8 |
| 3 | 5 | | 40 |
| 4 | 5 | | 200 |
| 5 | 5 | | 1000 |
| 6 | 5 | | 5000 |
| 7 | 5 | Murine | 8 |
| 8 | 5 | | 40 |
| 9 | 5 | | 200 |
| 10 | 5 | | 1000 |
| 11 | 5 | | 5000 |

Liquid Matrigel was maintained at 4° C. IL6 was added to Matrigel to the final concentration indicated, mixed thoroughly and stored overnight at 4° C. The injection sites were located on the dorsal side approximately 0.25 inches caudal to the last rib and 0.25 inches from the backbone on each side. The mice were injected in two sites with 0.5 mL of Matrigel. The area swelled if the injection was done properly.

Nude female mice (4-6 weeks old) obtained from Charles River (Raleigh, N.C.) were used in the study. Matrigel prepared from the Engelbreth-Holm-Swarm tumor was obtained from Becton Dickinson (Bedford, Mass.). C57 antibody (human IgG specific for CMV) from Centocor (Malvern, Pa.). Human IL6) and murine IL6 (mIL6) was purchased (R&D Systems, Minneapolis, Minn.). On day 1 of the study, 55 mice were randomized into 11 groups (n=5/group). Mice were anesthetized with Ketamine (80 mg/kg, IP). Animals were injected in two sites with 0.5 mL of Matrigel. On day 7 mice were euthanized by CO₂ asphyxiation. Plugs were surgically removed and weighed, photographed and graded for angiogenesis. Two plugs per animal were assayed for hemoglobin content using a Drabkin kit (Sigma, ST Louis, Mo.).

To measure the total area of neovessels, a computerized digitizer called the Phase 3 Image System was used. Photos were taken from both top surface and bottom surface of entire Matrigel plugs with 2× magnification objective of the inverted phase contrast microscope. The vessel length and number per field were calculated using the trace program of Phase 3 Image System. The mean value from all photos of entire Matrigel plugs was calculated with standard deviation from the mean.

To measure hemoglobin, the Matrigel plug was lysed with lysis buffer (1% SDS, 0.5% Triton). The hemoglobin content of the gels was quantitated using Drabkin reagent kit (Sigma, St. Louis, Mo.). The concentration of hemoglobin in the gels was determined from a standard curve of hemoglobin. Hemoglobin content was expressed as milligrams hemoglobin per gram Matrigel. Means±SEM were calculated using the Student's unpaired t test; p<0.05 was considered statistically significant.

IL6—Induced Angiogenesis in Matrigel

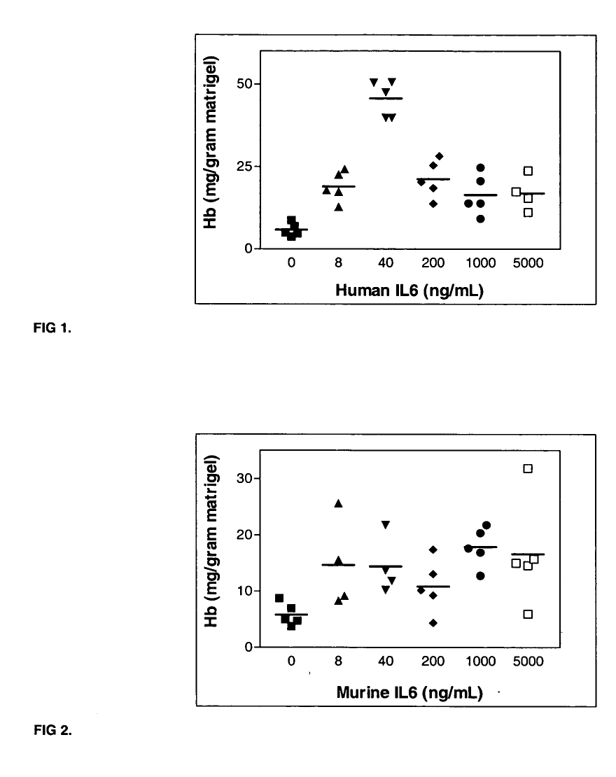

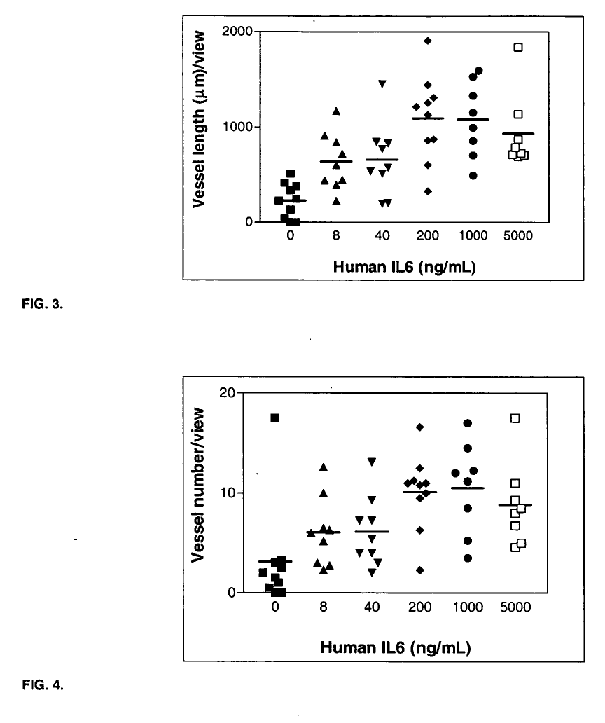

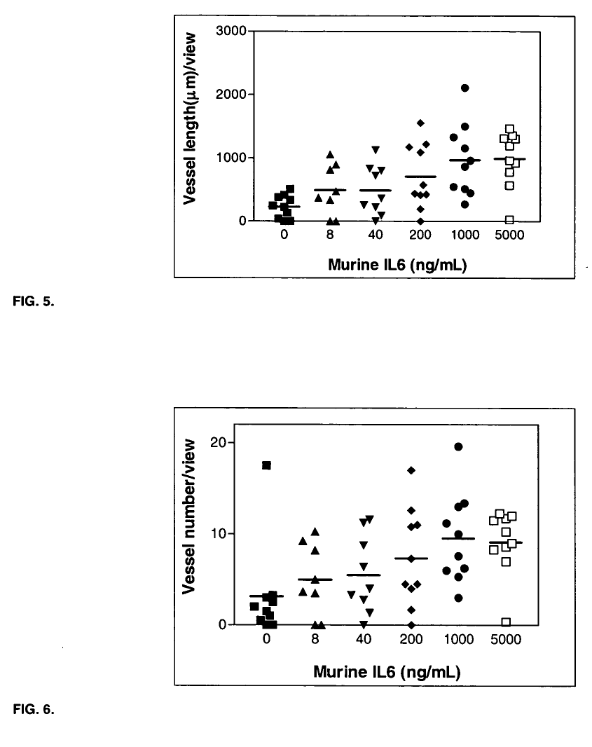

There was an observable difference in color and clearly visible vessels evident in excised Matrigel plugs in which had either mIL6 or hIL6 was administered as compared to those injected without added IL6. Photomicrographs at 2× enlargment documented the formation of extensive vessels (NOT SHOWN). Both human and murine IL6 increased the Hb content in the Matrigel plugs over that seen with no added cytokine (FIGS. 1, 2), as well as the vessel length and vessel number (FIG. 3-6). The maximal effect on vessel density and Hb content was at a concentration of about 200 ng/ml for both human IL6 and murine IL6 (FIGS. 1-6). The total Hb, vessel length, and vessel number in the IL6 groups was always significantly higher than in groups without IL6 (p<0.001) (FIGS. 1-6).

### EXAMPLE 2

### Inhibition of Angiogenesis In Vivo by Anti-IL6 Mab

In Study 2, animals received an IV injection of cCLB8 Mab, anti-human IL6, also called chimeric CLB8 (Centocor, Malvern, Pa.) or control antibody (C57) immediately following injection of Matrigel spiked with human IL6 at 200 ng/mL, as indicated in Table 2. On day 1 of the study, 42 mice were randomized into 7 groups (n=6/group). Mice were anesthetized with Ketamine (80 mg/kg, IP). Animals were injected in two sites with 0.5 mL of Matrigel. Antibodies (or vehicle) were injected IV immediately after Matrigel injections. On day 7 mice were euthanized by CO₂ asphyxiation. Plugs were surgically removed and weighed.

TABLE 2
The experimental design for Study 2.

| | | | | | Antibody | |
|---|---|---|---|---|---|---|
| | | Matrigel Containing: | | | (IV injection) | |
| Group | | hIL6 | cCLB8 | C57 | | Dose |
| # | n | (ng/mL) | (μg/mL) | (μg/mL) | | (mg/kg) |
| 1 | 6 | 0 | 0 | 0 | PBS | DVE* |
| 2 | 6 | 0 | 0 | 0 | | |
| 3 | 6 | 200 | 200 | 0 | | |
| 4 | 6 | 200 | 0 | 0 | cCLB8 | 10 |
| 5 | 6 | 200 | 200 | 0 | | |
| 6 | 6 | 200 | 0 | 0 | C57 | 10 |
| 7 | 6 | 200 | 0 | 200 | | |

*DVE: dose-volume equivalent, 10 mL/kg

Study 2 was performed using the same methods and materials as described in Study 1 except that, where indicated, the Matrigel was also mixed with an antibody and kept on ice.

cCLB8 Inhibited Angiogenesis

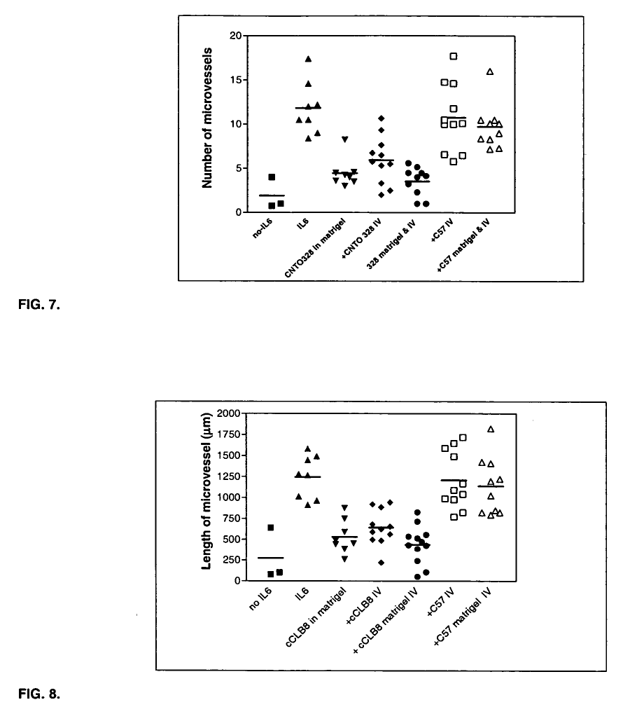

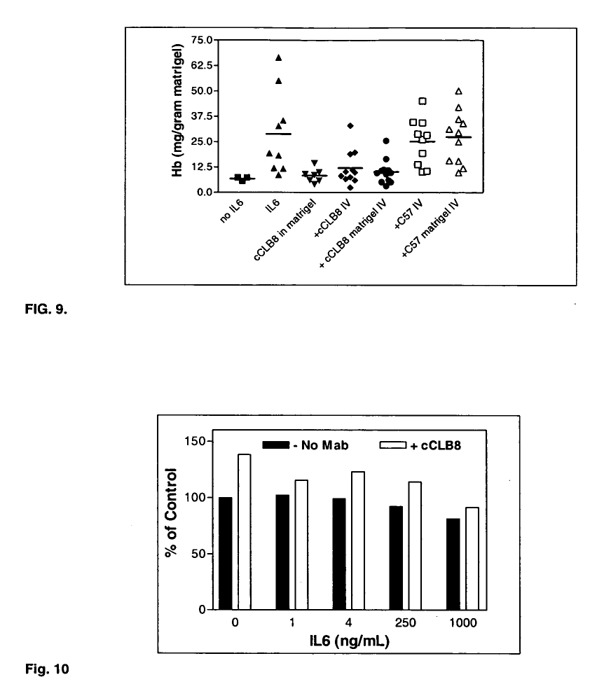

IL6 induced blood vessel formation in Matrigel plugs, and cCLB8 decreased the vessel formation induced by IL6. Matrigel plugs with IL6 incorporated have more red color than Matrigel plugs with no IL6, and C57 Matrigel plugs have more color than cCLB8 Matrigel plugs. Using vessel counting and Hb content, the results showed that IL6 significantly increased vessel formation in Matrigel plugs, and cCLB8 significantly inhibited vessel formation induced by IL6 (FIG. 7-9), whether cCLB8 was included in the Matrigel or injected IV after Matrigel plug injection.

Photomicrographs of Matrigel plugs at 2× magnification clearly documented the absence of vessels in the plugs from cCLB6 treated mice, similar to the control with no added IL6, while those plugs from mice treated with C57 had clearly discernable microvessels within them.

### EXAMPLE 3

### IL6 Suppression of Apoptosis Reversed by CCLB8

CNTO 95 (human IgG specific for αvβ3 and αvβ5 integrins) C57 (human IgG specific for CMV) and CLB8 were produced at Centocor (Malvern, Pa.). Human IL6 (hlL6) was obtained from R&D Systems (Minneapolis, Minn.). HUVEC, human umbilical vein endothelial cells, were purchased from Clonetics (Walkersville, Md.). HUVECs were cultured in EBM medium kit (Clonetics) containing 10% FCS, Long R Insulin-like Growth Factor-1, ascorbic acid, hydrocortisone, human EGF, hVEGF, hFGF-b, gentamicin sulfate, and amphotericin-B. Cells were incubated at 37° C. and 5% CO₂ and media was changed every 2 to 3 days. Passages 3 to 8 were used in all experiments.

DNA fragmentation was analyzed by the Cell Death Detection ELISA Kit (Roche Diagnostics GmBH, Mannheim, Germany) as recommended by the manufacturer. The assay is a quantitative sandwich-enzyme-immunoassays which uses mouse Mabs capable of detecting DNA and histones allowing for detection of mono- and oligonucleosomes in cell lysates. Cell apoptosis is directly proportional to the final absorbance at 405 nm. All determinations were performed in triplicate.

DNA fragment was measured in HUVEC cells after incubation in the presence of IL6 with and without CNTO 328 (10 μg/mL) for 3 days. The datas shown in FIG. 10 DNA are mean±SD of triplicate determinations, expressed as percent of control, with cells in serum free medium without IL6 or CNTO 328 defined as 100%. The experiment shows cCLB8 is capable of inducing apoptosis as measured by DNA fragmentation in HUVEC in the absence of IL6 and can reverse the protective effect of IL6 seen at high concentrations.

### EXAMPLE 4

### IL6 Induced Migration of HUVEC

HUVEC as described and cultured in the previous example. Sub-confluent 24-hr cell cultures of HUVECS were starved with serum free medium overnight, harvested with trypsin-EDTA, washed twice, and resuspended in serum free media containing 0.1% BSA. Cells (100,000/500 microL) were added to the upper chamber. To facilitate chemotactic cell migration, 750 microL of medium containing 0.1% BSA and different concentrations of IL6 or cCLB8 was added to the bottom chambers and the plate was placed in a tissue culture incubator. Migration was terminated after the specified elapsed time by removing the cells in the upper chamber with a cotton swab. The filters were fixed with 3% paraformaldehyde and stained with Crystal Violet. The extent of cell migration was determined by light microscopy; images were analyzed using the Phase 3 image analysis software (Glen Mills, Pa.). The software analyzes the total area occupied by the stained cells on the bottom side of the filter, which is directly proportional to the extent of cell migration. The stained transwells were destained with 10% acetic acid, and the absorbance was recorded with 590 nm.

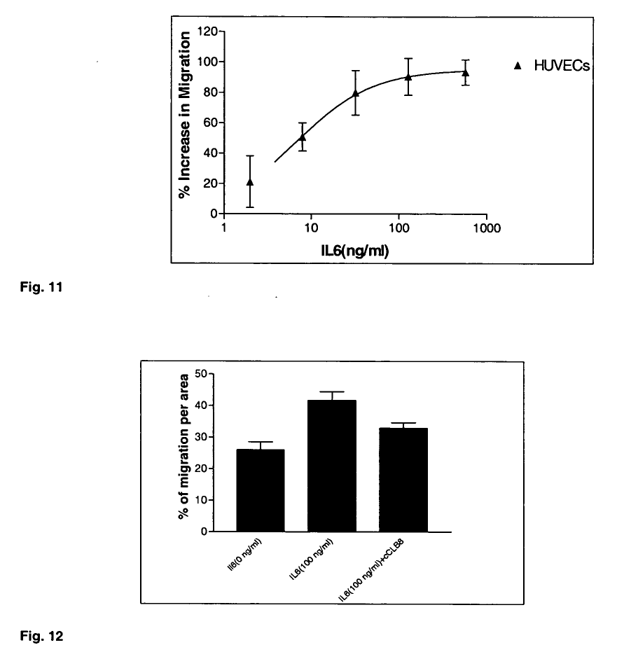

The undersides of migration chamber filters were coated with 0.5 microg/mL vitronectin, and the assay was performed as described in methods. Cells were allowed to migrate for 6 h. The undersides of migration chamber filters were coated with 0.5 microg/mL vitronectin, and the assay was performed as described in methods. Cells were allowed to migrate for 6 h. Each data point is the mean±SD of 3 transwell filters (FIG. 11-12). The data in FIG. 11 show a dose dependent response of the HUVEC cells to IL-6 with maximal activity at about 100 ng/ml. In the presence of a neutralizing anti-IL6 Mab, cCLB8, amount of migration is suppressed.

### EXAMPLE 5

### Effect of Anti-IL6 Mab on IL6 Induced Survival of Endothelial Cells

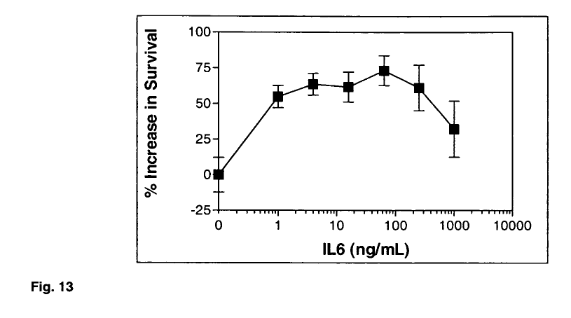

HUVEC as described and cultured in the previous two examples. Cell survival and proliferation were measured in similar assays using commercially available kits. Briefly, 6000 cells/well were seeded in a 96-well microplate and fed complete medium. After 18 hr, cells were rinsed twice and incubated with serum-free media for 24 hrs. Then recombinant IL6 and antibodies were added in serum free medium. Cells were cultured for 48 hrs. Extent of cell survival was determined by the MTS kit (Promega, Madison, Wis.). For the MTS assay absorbence was measured at 490 nm. The results were expressed as a percentage of the value by cells in serum free media with no IL6. All determinations were performed in triplicate wells. These results (FIG. 13) demonstrate that IL6 has a direct effect on endothelial survival under conditions of limited nutrition. Such conditions are found in rapidly neovascularizing tissues such as that in growing tumors and in damaged skin or the eye.

### SUMMARY

The experiments described herein demonstrate that IL6 induced angiogenesis and related functions of endothelial cells which are stimulated by IL6 can be reduced by a specific Mab that prevents IL6 signaling through a receptor complex which includes gp130.

The process of angiogenesis as it occurs in new tissue forming in vivo was simulated in Matrigel plugs in nude mice, and as measured by increased number and length of microvessels, and increased Hb content. The major component is laminin, but Matrigel also contains trace amounts of fibroblast growth factor, TGF-beta, tissue plasminogen activator, and other growth factors that occur naturally in the EHS tumor. Matrigel is the basis for several types of tumor cell invasion assays and provides the necessary substrate for the study of angiogenesis. Matrigel forms a soft gel plug when injected subcutaneously into mice or rats and supports an intense vascular response when supplemented with angiogenic factors.

cCLB8 inhibited angiogenesis in Matrigel when it was incorporated in the Matrigel.

Experimental results demonstrated that a single injection of cCLB8 almost completely inhibited IL-6 mediated angiogenesis in the Matrigel plug model in nude mice. In addition, cCLB8 inhibited angiogenesis in Matrigel when it was injected IV following injection of Matrigel.

1. A method for treating an angiogenesis-dependent disease in a mammal in need thereof comprising administering to the mammal an IL-6 antagonist which prevents IL6 activation of signaling through membrane bound receptors in an amount effective to inhibit angiogenesis in said mammal.

2. The method of claim 1 wherein the 11-6 antagonist is and IL-6 monoclonal antibody or a fragment thereof.

3. The method according to claim 2, in which the antibody fragment is an Fab, Fab′, or F(ab′)₂ fragment or derivative thereof.

4. The method according to claim 2, in which the monoclonal antibody competes with monoclonal antibody cCLB8 for binding to human IL6.

5. The method according to claim 2, in which the monoclonal antibody is administered intravenously.

6. The method according to claim 2, in which the monoclonal antibody is administered in the amount of from 0.05 mg/kg to 12.0 mg/kg body weight.

7. The method according to claim 2, in which the monoclonal antibody is administered in a bolus dose followed by an infusion of said antibody.

8. The method according to claim 1, in which the mammal is a human patient.

9. The method according to claim 2, in which said monoclonal antibody treats cancer.

10. The method of claim 1, wherein the angiogenesis-dependent diseases is a disease selected from the group consisting of cancer metastasis, angioma, angiofibroma, diabetic retinopathy, premature infant's retinopathy, neovascular glaucoma, corneal disease induced by angiogenesis, involutional macula, macular degeneration, pterygium, retinal degeneration, retrolental fibroplasias, granular conjunctivitis, psoriasis, telangiectasis, pyogenic granuloma, seborrheic dermatitis, acne and arthritis.

11. The method according to claim 1, in which said angiogenesis dependent disease is an inflammatory disease selected from the group consisting of rheumatoid arthritis, macular degeneration, psoriasis, diabetic retinopathy.

12. The method according to claim 1, in which said angiogenesis dependent disease is an angiogenic skin disorder selected from the group consisting of psoriasis, venous ulcers, acne, rosacea, warts, eczema, hemangiomas, and lymphangiogenesis.

13. The method according to claim 1, in which said angiogenesis dependent disease is a disorder involving corneal or retinal neovascularization.

14. A method for inhibiting tumor growth in a mammal in need thereof comprising administering to the mammal a monoclonal antibody or fragment thereof which prevents IL6 activation of signaling through membrane bound receptors in an amount effective to inhibit the growth of said tumor.

15. A method for preventing tumor growth in a mammal in need thereof comprising administering to the mammal a monoclonal antibody or fragment thereof which prevents IL6 activation of signaling through membrane bound receptors in an amount effective to prevent the growth of said tumor in said mammal.

16. A method for preventing metastases in a mammal in need thereof comprising administering to the mammal a monoclonal antibody or fragment which prevents IL6 activation of signaling through membrane bound receptors in an amount effective to prevent metastases in said mammal.

17. A method of any of claims 2, 13, 14, or 15 wherein the antibody is cCLB8 or a fragment thereof.

18. A method of any of claims 1, 13, 14, or 15 where in the antibody is administered in combination with a second anti-angiogenic agent.

19. A method of claim 17 where the second anti-angiogenic agent is a Mab capable of specifically binding the adhesion molecules containing alphaV.
